(19) United States
(12) Patent Application Publication
(10) Pub. No.: US 2020/0367834 A1
(43) Pub. Date: Nov. 26, 2020

(54) DEVICE FOR PREDICTING BODY WEIGHT OF A PERSON AND DEVICE AND METHOD FOR HEALTH MANAGEMENT

(71) Applicant: OMRON Corporation, KYOTO (JP)

(72) Inventor: Naoki TSUCHIYA, Shinagawa-ku, TOKYO (JP)

(73) Assignee: OMRON Corporation, KYOTO (JP)

(21) Appl. No.: 16/968,157

(22) PCT Filed: Mar. 5, 2018
(86) PCT No.: PCT/IB2018/051391

§ 371 Date: Aug. 6, 2020

(51) Int. Cl. A61B 5/00; A61B 5/022; G06N 3/08; G16H 50/20

(52) U.S. Cl. CPC A61B 5/7275; A61B 5/022; G16H 50/20; G06N 3/08; A61B 5/4869

(57) ABSTRACT

The present disclosure provides a device for predicting body weight of a person and device and method for health management. The device for predicting body weight of a person according to the present disclosure includes an acquisition unit for obtaining blood pressure of each person in multiple persons; an input unit for inputting body weights of the persons; a storage unit for associatively storing the blood pressure and the corresponding body weights; a combination unit for obtaining average blood pressure and obtaining an average body weight, wherein the average blood pressure and the average body weight are associatively stored in the storage unit as learning data; and a prediction unit for performing machine learning on the basis of the learning data and predict body weight. The organism data is processed to an extent that makes the detected persons unidentifiable.

### TECHNICAL FIELD

The present application relates to the field of biometric identification. More particularly, the present application relates to a device for predicting body weight of a person and device and method for health management.

### BACKGROUND

For existing identification devices, an organism-data-based machine learning method is utilized to generate an identification device with a predetermined capability according to input organism data and corresponding identification results, wherein, according to the identification capability which may be achieved, the organism data for input into the identification device sometimes includes data of persons or identifiable individuals determined as objects of the collected organism data. For example, in a present solution, blood pressure or body weight of a person is stored for machine learning, and it can be used to identify the biological characteristics of the person. That is to say, the biological characteristics (blood pressure or body weight) of the person can be obtained by others. For the organism data, it is necessary to make these objects, persons or individuals unidentifiable from the organism data sometimes.

Under this condition, a method for performing encryption by researching an expression manner of organism data is proposed. In a cancelable biometric identification technology, organism information is irreversibly converted by a conversion parameter, and the converted information is stored in a system as a registration template. During compared identification, compared organism information is converted in the same manner by the same conversion parameter, and is compared with the registration template, thereby implementing data authentication and identification. However, if learning data provided for an identification device is independently encrypted, compound processing is required to be performed every time when a learner is generated, and no learner may be smoothly generated sometimes.

Therefore, it is necessary to provide a technology capable of ensuring anonymity of data without encrypting the learning data.

### SUMMARY

### The Technical Problem to be Solved

The present disclosure is intended to solve at least part or all of the foregoing problems.

### Means of Solving the Technical Problem

In embodiments of the present disclosure, organism information and corresponding event information are acquired, the organism information is combined, moreover, the event information is combined, and the combined organism information and the combined event information are adopted as learning data of an identification device.

The embodiments of the present disclosure provide a device and method for health management, so as to at least solve the problem of how to process organism information of learning data for training an identification device into information from which identification information, such as organism information, of objects, persons or individuals may not be acquired.

According to one aspect of the embodiments of the present disclosure, a device for predicting body weight of a person is provided, which includes an acquisition unit, an input unit, a storage unit, a combination unit, and a prediction unit. The acquisition unit is configured to obtain blood pressure of each person in multiple persons. The input unit is configured to input body weights of the persons when the blood pressure is obtained. The storage unit is configured to associatively store the blood pressure and the corresponding body weights. The combination unit is configured to obtain average blood pressure of the blood pressure of at least two persons and obtain an average body weight of the body weights of the at least two persons, wherein the average blood pressure and the average body weight are associatively stored in the storage unit as learning data. And the prediction unit is configured to perform machine learning on the basis of the learning data and predict body weight according to blood pressure on the basis of a learning result.

In such a manner, the blood pressure and body weights of the persons included in the learning data are combined and stored, so that it is impossible to directly obtain identification information of the persons from the blood pressure and the body weights. Correct body weight prediction may be performed according to the processed learning data.

According to another aspect of the embodiments of the present disclosure, a device for health management is provided, which includes an acquisition unit, an input unit, a storage unit, a combination unit, and a prediction unit. The acquisition unit is configured to obtain organism information of each person in multiple persons. The input unit is configured to obtain event information corresponding to each piece of organism information, the event information representing a physical entity parameter obtained by means of a sensing device. The storage unit is configured to associatively store the organism information and the corresponding event information. The combination unit is configured to obtain a combination value of combination of at least two pieces of organism information and obtain combined event information of combination of the event information respectively corresponding to the at least two pieces of organism information, wherein the combination value and the combined event information are associatively stored in the storage unit as learning data. And the prediction unit is configured to perform machine learning on the basis of the learning data and predict event information according to organism information on the basis of a learning result.

In such a manner, the organism information and corresponding event information included in the learning data are combined and stored, so that it is impossible to directly obtain identification information, such as organism information, of objects or persons from the organism information. Correct event prediction may be performed according to the processed learning data.

According to an exemplary embodiment of the present disclosure, wherein the event information is information in a numerical form, the device for health management further including a label generation unit. The label generation unit is configured to calculate an average value of the event information corresponding to the at least two pieces of organism information as the combined event information; and assign the combined event information to the combination value as a label.

Therefore, the event information in the numerical form is combined and processed, and it is impossible to directly obtain the identification information, used as the organism information, of the objects or the persons from the event information.

According to an exemplary embodiment of the present disclosure, wherein the event information is information representing an action, the label generation unit is configured to determine the event information of which an occurrence frequency is higher than the other event information in the event information corresponding to the at least two pieces of organism information as the combined event information; and assign the combined event information to the combination value as a label.

Therefore, the event information representing the action is combined and processed, and it is impossible to directly obtain the identification information, used as the organism information, of the objects or the persons from the event information.

According to an exemplary embodiment of the present disclosure, the combination value is an average value of the at least two pieces of organism information.

Therefore, the organism information is combined and processed, and it is impossible to directly obtain the identification information, used as the organism information, of the objects or the persons from the organism information.

According to an exemplary embodiment of the present disclosure, the device for health management further includes an encrypted information generation unit. The encrypted information generation unit is configured to add a random number to each of the at least two pieces of organism information to obtain multiple pieces of encrypted organism information, wherein an average value of at least two pieces of encrypted organism information is equal to the average value of the at least two pieces of organism information, and moreover, the encrypted information generation unit sends the multiple pieces of encrypted organism information to the storage unit to replace the organism information associated with the event information in the stored learning data with the corresponding encrypted organism information.

Therefore, the organism information is further encrypted, the organism information is changed at the same time of keeping the required learning data, and it is impossible to directly obtain the identification information, used as the organism information, of the objects or the persons from the organism information.

According to an exemplary embodiment of the present disclosure, at least two persons corresponding to the at least two pieces of organism information for combination are persons with the same event information.

Therefore, the organism information suitable for the learning data is selected as learning data.

According to an exemplary embodiment of the present disclosure, for the at least two persons corresponding to the at least two pieces of organism information for combination, closeness between values of the at least two pieces of organism information is higher than a predetermined threshold value.

Therefore, the organism information suitable for the learning data is selected as the learning data.

According to another aspect of the embodiments of the present disclosure, a method for health management is provided, which includes: organism information of each person in multiple persons is obtained; event information corresponding to each piece of organism information is obtained, the event information representing a physical entity parameter obtained by means of a sensing device; the organism information and the corresponding event information are associatively stored; a combination value of combination of at least two pieces of organism information is obtained, and combined event information of combination of the event information respectively corresponding to the at least two pieces of organism information is obtained, wherein the combination value and the combined event information are associatively stored as learning data, and performing machine learning on the basis of the learning data and predict event information according to organism information on the basis of a learning result.

In such a manner, the organism information and corresponding event information included in the learning data are combined and stored, so that it is impossible to directly obtain identification information, such as organism information, of objects or persons from the organism information. Correct event prediction may be performed according to the processed learning data.

According to an exemplary embodiment of the present disclosure, wherein the event information is information in a numerical form, the method for health management further including: an average value of the event information corresponding to the at least two pieces of organism information is calculated as the combined event information; and the combined event information is assigned to the combination value as a label.

Therefore, the event information in the numerical form is combined and processed, and it is impossible to directly obtain the identification information, used as the organism information, of the objects or the persons from the event information.

According to an exemplary embodiment of the present disclosure, wherein the event information is information representing an action, the method for health management further including: the event information of which an occurrence frequency is higher than the other event information in the event information corresponding to the at least two pieces of organism information is determined as the combined event information; and the combined event information is assigned to the combination value as a label.

Therefore, the event information representing the action is combined and processed, and it is impossible to directly obtain the identification information, used as the organism information, of the objects or the persons from the event information.

According to an exemplary embodiment of the present disclosure, the combination value is an average value of the at least two pieces of organism information.

Therefore, the organism information is combined and processed, and it is impossible to directly obtain the identification information, used as the organism information, of the objects or the persons from the organism information.

According to an exemplary embodiment of the present disclosure, the method for health management further includes: a random number is added to each of the at least two pieces of organism information to obtain multiple pieces of encrypted organism information, wherein an average value of at least two pieces of encrypted organism information is equal to the average value of the at least two pieces of organism information, and moreover, the organism information associated with the event information in the stored learning data is replaced with the corresponding encrypted organism information.

Therefore, the organism information is further encrypted, the organism information is changed at the same time of keeping the required learning data, and it is impossible to directly obtain the identification information, used as the organism information, of the objects or the persons from the organism information.

According to an exemplary embodiment of the present disclosure, at least two persons corresponding to the at least two pieces of organism information for combination are persons with the same event information.

Therefore, the organism information suitable for the learning data is selected as learning data.

According to an exemplary embodiment of the present disclosure, for the at least two persons corresponding to the at least two pieces of organism information for combination, closeness between values of the at least two pieces of organism information is higher than a predetermined threshold value.

Therefore, the organism information suitable for the learning data is selected as the learning data.

According to another aspect of the embodiments of the present disclosure, a method for health management is provided, which includes: blood pressure of each person in multiple persons is obtained; body weights of the persons when the blood pressure is obtained are obtained; the blood pressure and the corresponding body weights are associatively stored; and average blood pressure of the blood pressure of at least two persons is obtained, and an average body weight of the body weights of the at least two persons is obtained, wherein the average blood pressure and the average body weight are associatively stored as learning data.

According to another aspect of the embodiments of the present disclosure, an event prediction method is provided, which includes: learning data generated by the foregoing method for health management is obtained, machine learning is performed on the basis of the learning data, and body weight is predicted according to blood pressure on the basis of a learning result.

Therefore, correct body weight prediction may be performed according to the processed learning data.

According to another aspect of the embodiments of the present disclosure, a storage medium is provided, on which a program is stored, the program, when being executed, enabling equipment including the storage medium to execute the foregoing method.

According to another aspect of the embodiments of the present disclosure, a terminal is provided, which includes: one or more processors; a memory; a display device; and one or more programs, wherein the one or more programs are stored in the memory, and are configured to be executed by the one or more processors, and the one or more programs are configured to execute the foregoing method.

The program and the storage medium may achieve the same effect as each foregoing method.

### Technical Effect

In the embodiments of the present disclosure, the processed learning data may correctly train the identification device or be configured for event prediction, and meanwhile, it is impossible to identify the identification information, used as the organism, of the objects or the persons from the processed learning data.

### BRIEF DESCRIPTION OF THE DRAWINGS

The drawings described herein are used to provide a further understanding of the present disclosure and constitute a part of the present disclosure. The schematic embodiments of the present disclosure and the descriptions thereof are used to explain the present disclosure, and do not constitute improper limitations to the present disclosure. In the drawings:

FIG. 1 is a mode diagram of a hardware structure of a system for health management 100 according to an implementation mode of the present disclosure;

FIG. 2 is a block diagram of a device for health management according to an embodiment of the present disclosure;

FIG. 3 is a data distribution diagram of organism information and encrypted organism information according to an embodiment of the present disclosure;

FIG. 4 is a flowchart of a method for health management according to an embodiment of the present disclosure;

FIG. 5 is a flowchart of a method for health management according to an exemplary embodiment of the present disclosure;

FIG. 6 is a flowchart of a method for health management according to an exemplary embodiment of the present disclosure; and

FIG. 7 is a flowchart of a method for health management according to an exemplary embodiment of the present disclosure.

### DETAILED DESCRIPTION

In sequence to make those skilled in the art better understand the solutions of the present disclosure, the technical solutions in the embodiments of the present disclosure are clearly and completely described below in combination with the accompanying drawings in the embodiments of the present disclosure. Apparently, the described embodiments are merely a part of the embodiments of the present disclosure, rather than all of the embodiments. All other embodiments obtained by those skilled in the art based on the embodiments in the present disclosure, without creative efforts, shall fall within the protection scope of the present disclosure.

It is important to note that terms “first”, “second” and the like in the specification, claims and drawings of the present disclosure are adopted not to describe a specific sequence or order but to distinguish similar objects. It should be understood that data used like this may be exchanged under a proper condition for implementation of the embodiments of the present disclosure described herein in a sequence besides those shown or described herein. In addition, terms “include” and “have” and any transformation thereof are intended to cover nonexclusive inclusions. For example, a process, method, system, product or equipment including a series of steps or modules or units is not limited to those steps or modules or units which are clearly listed, but may include other steps or modules or units which are not clearly listed or intrinsic to the process, the method, the product or the equipment.

In the technical solutions of the present disclosure, organism information obtained from detected persons determined as objects of the organism information is combined between the organism information of multiple detected persons as learning data. The combined organism data is used as the learning data, so that a learner may be generated for training of an identification device or for event prediction according to the learning data of the identifiable detected persons.

At first, a hardware structure of a system for health management 100 according to an implementation mode of the present disclosure is described.

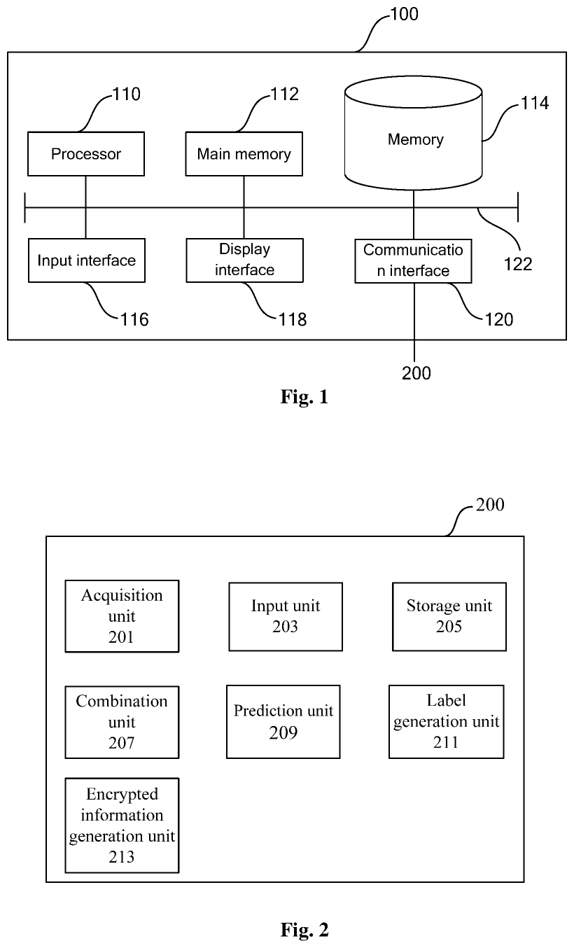

FIG. 1 is a mode diagram of a hardware structure of a system for health management 100 according to an implementation mode of the present disclosure. As shown in FIG. 1, for example, the system for health management 100 may be implemented by a general purpose computer. The system for health management 100 may include a processor 110, a main memory 112, a memory 114, an input interface 116, a display interface 118 and a communication interface 120. These parts may, for example, communicate with one another through an internal bus 122.

The processor 110 extends a program stored in the memory 114 on the main memory 112 for execution, thereby realizing functions and processing described hereinafter. The main memory 112 may be structured to be a nonvolatile memory, and plays a role as a working memory required by program execution of the processor 110.

The input interface 116 may be connected with an input unit such as a mouse and a keyboard, and receives an instruction input by operating the input unit by an operator.

The display interface 118 may be connected with a display, and may output various processing results generated by program execution of the processor 110 to the display.

The communication interface 120 is configured to communicate with a Programmable Logic Controller (PLC), a database device and the like through a network 200.

The memory 114 may store a program capable of determining a computer as the system for health management 100 to realize functions, for example, a program for health management and an Operating System (OS).

The program for health management stored in the memory 114 may be installed in the system for health management 100 through an optical recording medium such as a Digital Versatile Disc (DVD) or a semiconductor recording medium such as a Universal Serial Bus (USB) memory. Or, the program for health management may also be downloaded from a server device and the like on the network.

The program for health management according to the implementation mode may also be provided in a manner of combination with another program. Under such a condition, the program for health management does not include a module included in the other program of such a combination, but cooperates with the other program for processing. Therefore, the program for health management according to the implementation mode may also be in a form of combination with the other program.

According to one embodiment of the present disclosure, a device for health management is provided. FIG. 2 is a block diagram of a device for health management according to an embodiment of the present disclosure. As shown in FIG. 2, the device for health management 200 includes: an acquisition unit 201, configured to obtain blood pressure of each person in multiple persons; an input unit 203, configured to obtain body weight when obtaining blood pressure; a storage unit 205, configured to associatively store the blood pressure and the corresponding body weight; a combination unit 207, configured to obtain an average blood pressure of blood pressure of at least two persons. and obtain an average body weight of the body weight of at least two persons, wherein the average blood pressure and the average body weight are associatively stored in the storage unit as learning data. The learning data are generated for machine learning. The prediction unit 209, for example, when performing machine learning on the basis of the learning data, may link certain blood value with certain body weight. And the prediction unit 209 is configured to predict body weight according to blood pressure on the basis of a learning result.

In another embodiment, the machine learning can be performed by neural network, such that the neural network is capable of predicting body weight from blood pressure. The neural network may be any kind of existing neural network, which may receive input as learning data, e.g., a set of data that includes data used for prediction and the corresponding prediction result. The neural network, after the received learning data are processed, may be used for prediction. For example, blood pressure and the corresponding body weight are received as learning data, and the neural network performs machine learning. That is to say the neural network creates links between certain blood pressure and body weight. After machine learning, the neural network is trained, and may receive further input for prediction. If blood pressure is received, the neural network may generate a prediction result relating what is the corresponding body weight or it can generate a possibility value about possible body weight based on the created links.

The acquisition unit 201 is, for example, the apparatus for acquiring blood pressure, and may also be equipment acquiring the blood pressure from the corresponding apparatus. The acquisition unit 201 obtains blood pressure of multiple persons for subsequent processing. For obtaining effective learning data, besides the blood pressure, the body weight when obtaining the blood pressure is also required. The learning data indicates body weight when the blood pressure is acquired.

The input unit 203 is configured to acquire the body weight. The input unit 203 may be equipment for manually inputting the body weight by a user, and may also acquire the body weight from other equipment.

The storage unit 205 is, for example, a nonvolatile memory, or any memory capable of storing data, wherein the blood pressure and the corresponding body weight are associatively stored.

The combination unit 207 is configured to process the acquired blood pressure and the body weight. The blood pressure and body weight processed by the combination unit 207 may be configured to accurately train the identification device or for event prediction as the learning data, and it is impossible to identify information of the persons from the processed data. For the blood pressure, the combination unit 207 computes and obtains the average blood pressure and average body weight. In such a manner, the average blood pressure and the average body weight do not directly represent the information of the persons, but represent changed information, so that it is impossible to identify the person from whom the organism information is acquired. In other words, it is generated a blood pressure and a corresponding body weight for a virtualized person. And the generated data cannot be used to determine an actual person. The combination unit 207 sends the processed average blood pressure and the average body weight to the storage unit 205, and the storage unit 205 stores the average blood pressure and the average body weight in an association manner, that is, the average blood pressure corresponds to the average body weight.

The event prediction unit 209 may be an identification device, may be trained by the learning data, and may also determine the body weight of the persons when the blood pressure is input into it.

In another embodiment, the machine learning and prediction can be used for industrial usage, for example, factory automation. In such a case, for example, the learning data includes heart rates and corresponding processes that persons are currently performing or have just performed. In particular, the acquisition unit 201 obtains heart rates of a plurality of persons performing industrial process. The input unit 203 may be used for entering the processes which the persons are currently performing or have just performed. That is, certain heart rate is related to a certain process. There may be multiple processes. For each kind of process, the corresponding heart rates may form a data set. For multiple kinds of processes, there are corresponding numbers of data sets. To convert the heart rates and processes to the extent that they cannot be used to identify a person, the combination unit 207 generate an average heart rate for the heart rates in each data set. It is seen that an average heart rate does not represent the heart rate of any one of the persons and cannot be used to identify a person. Accordingly, each average heart rate corresponds to a certain kind of process. The average heart rates and corresponding processes may be associatively stored by the storage unit 205 as learning data for machine learning. The prediction unit 209 may receive the learning data and perform machine learning on the basis of the average heart rates and corresponding processes. After the machine learning, the prediction unit 209 is capable of predicting processes that persons are currently performing or have just performed on the basis of inputs of heart rates. If a heart rate is detected or received by the prediction unit 209, it generates the corresponding process that a person is currently performing or has just performed, or it generates the possibility for each possible process. In this embodiment, the processes which persons are performing or have just performed may be monitored.

According to another embodiment of the present disclosure, the learning data includes organism information and event information. The acquisition unit 201 is configured to obtain organism information of each person in multiple persons. The input unit 203 is configured to obtain event information corresponding to each piece of organism information. The storage unit 205 is configured to associatively store the organism information and the corresponding event information. And the combination unit 207 is configured to obtain a combination value of combination of at least two pieces of organism information and obtain combined event information of combination of the event information respectively corresponding to the at least two pieces of organism information, wherein the combination value and the combined event information are associatively stored in the storage unit as learning data. The prediction unit 209 is configured to obtain learning data generated and performs machine learning on the basis of the learning data. The prediction unit 209 predicts event information according to organism information on the basis of a learning result.

The organism information is information that indicates the biological characteristic of the persons. The persons for acquisition of the organism information are, for example, persons, and may also be other organisms. The organism information may be acquired from the persons through a corresponding apparatus. For example, data such as blood pressure and a heart rate may be acquired as organism information. It should be understood that other organism information may also be acquired, as long as the information may be used to train an identification device or for event prediction as learning data.

The acquisition unit 201 is, for example, the corresponding apparatus acquiring the organism information, and may also be equipment acquiring the organism information from the corresponding apparatus. The acquisition unit 201 obtains multiple pieces of organism information for subsequent processing. For obtaining effective learning data, besides the organism information, the event information corresponding to the organism information is also required. For example, the event information is information representing events occurring to the corresponding persons when the organism information is obtained, representing a physical entity parameter obtained by means of a sensing device, and the learning data indicates that the corresponding events occur to the persons when the organism information is acquired.

The input unit 203 is configured to acquire the event information. The input unit 203 may be equipment for manually inputting the event information by a user, and may also acquire the event information from other equipment.

The storage unit 205 is, for example, a nonvolatile memory, or any memory capable of storing data, wherein the organism information and the corresponding event information are associatively stored.

The combination unit 207 is configured to process the acquired organism information and the event information. The organism information and event information processed by the combination unit 207 may be configured to accurately train the identification device or for event prediction as the learning data, and it is impossible to identify information of the persons from the processed data. For the multiple pieces of organism information, the combination unit 207 combines the multiple pieces of organism information to obtain the combination value by a predetermined algorithm, and moreover, for multiple pieces of corresponding event information, the combination unit 207 combines the multiple pieces of event information by the predetermined algorithm. In such a manner, the combination value and the combined event information do not directly represent the organism information of the persons, but represent changed organism information, so that it is impossible to identify the person from whom the organism information is acquired. The combination unit 207 sends the processed combination value and combined event data to the storage unit 205, and the storage unit 205 stores the combination value and the combined event data in an association manner, that is, the combination value corresponds to the combined event information.

The foregoing device for health management 200 generates the learning data on the basis of the combination value and the combined event data, and the event prediction unit 209 may be an identification device, may be trained by the learning data, and may also determine the event information of the persons when the organism information, for example, the blood pressure and the heart rates, is input into it.

In such a manner, the organism information and corresponding event information included in the learning data are combined and stored, so that it is impossible to directly obtain identification information, such as organism information, of objects or persons from the organism information. Correct event prediction may be performed according to the processed learning data.

As shown in FIG. 2, according to an exemplary embodiment of the present disclosure, the device for health management 200 further includes: a label generation unit 211, configured to calculate an average value of the event information corresponding to the at least two pieces of organism information as the combined event information, wherein the event information is information in a numerical form; and assign the combined event information to the combination value as a label.

The label generation unit 211 is configured to assign the label to the combination value to represent the information corresponding to the combination value. For example, in an implementation mode, the event information is information in the numerical form, and for example, is body weights of the persons, and the label generation unit 211 calculates an average value of the information of the multiple persons in the numerical form as the combined event information, for example, an average body weight. The label generation unit 211 assigns the combined event information in an average value form to the combination value as corresponding information to represent that the combination value corresponds to the average value of the combined event information.

Therefore, the event information in the numerical form is combined and processed, and it is impossible to directly obtain the identification information, used as the organism information, of the objects or the persons from the event information.

According to an exemplary embodiment of the present disclosure, the label generation unit 211 is configured to determine the event information of which an occurrence frequency is higher than the other event information in the event information corresponding to the at least two pieces of organism information as the combined event information, wherein the event information is information representing an action; and assign the combined event information to the combination value as a label.

The event information may be information representing an action, besides the information in the numerical information such as the body weights of the persons. For example, the event information may be information representing sitting, standing, walking, jumping and the like of the persons. In case of use as learning data, the specific actions finished by the persons in these actions may be determined according to the corresponding organism information. Therefore, the event information of which the occurrence frequency is higher than the other actions may be selected from the multiple actions as the combined event information. Similarly, the combined event information does not represent identification information of a certain person anymore, but may be used to correctly train the identification device or for event prediction. The label generation unit 211 assigns the combined event information to the combination value as a label to represent that the combination value corresponds to the event information of which the occurrence frequency is higher than the other event.

Therefore, the event information representing the action is combined and processed, and it is impossible to directly obtain the identification information, used as the organism information, of the objects or the persons from the event information.

According to an exemplary embodiment of the present disclosure, the combination value is an average value of the at least two pieces of organism information. The organism information is, for example, numerical information of blood pressure, a heart rate and the like, and their average value may be adopted as the combination value. The combination value may be used as organism information in the learning data, and meanwhile, the combination value may not be used to acquire the identification information of the persons.

Therefore, the organism information is combined and processed, and it is impossible to directly obtain the identification information, used as the organism information, of the objects or the persons from the organism information.

As shown in FIG. 2, according to an exemplary embodiment of the present disclosure, the device for health management 200 further includes: an encrypted information generation unit 213, configured to add a random number to each of the at least two pieces of organism information to obtain multiple pieces of encrypted organism information, wherein an average value of at least two pieces of encrypted organism information is equal to the average value of the at least two pieces of organism information, and moreover, the encrypted information generation unit sends the multiple pieces of encrypted organism information to the storage unit to replace the organism information associated with the event information in the stored learning data with the corresponding encrypted organism information.

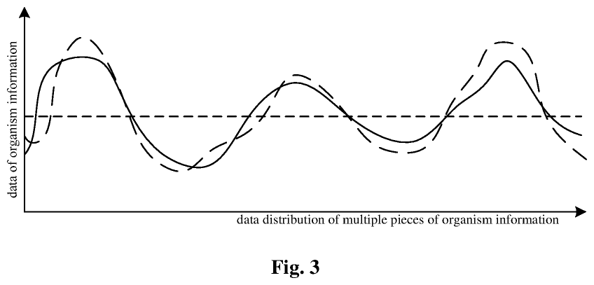

The combination value of the organism information may be further processed to be encrypted to make it further difficult to acquire the identification information of the persons. FIG. 3 is a data distribution diagram of organism information and encrypted organism information according to an embodiment of the present disclosure. As shown in FIG. 3, the ordinate axis represents data of the organism information, and the abscissa axis represents a data distribution of the multiple pieces of organism information. The multiple pieces of organism information are distributed on the two sides by taking the combination value as a centerline. By processing of the encrypted information generation unit 213, i.e., addition of the random number to the multiple pieces of organism information, each piece of organism information is changed, but an overall distribution of the data of the multiple pieces of organism information still takes the combination value as the centerline, that is, the combination value is not changed, but the separate organism information has been changed, so that it is impossible to acquire the identification information of the persons therefrom.

Therefore, the organism information is further encrypted, the organism information is changed at the same time of keeping the required learning data, and it is impossible to directly obtain the identification information, used as the organism information, of the objects or the persons from the organism information.

According to an exemplary embodiment of the present disclosure, at least two persons corresponding to the at least two pieces of organism information for combination are persons with the same event information.

In an exemplary embodiment, the persons are selected on the basis of a predetermined condition to provide reasonable samples used as the learning data. The persons are persons the same event occurs to when the organism information is acquired, so that the acquired data may be configured to determine the event occurring to the persons when the organism information is acquired.

Therefore, the organism information suitable for the learning data is selected as learning data.

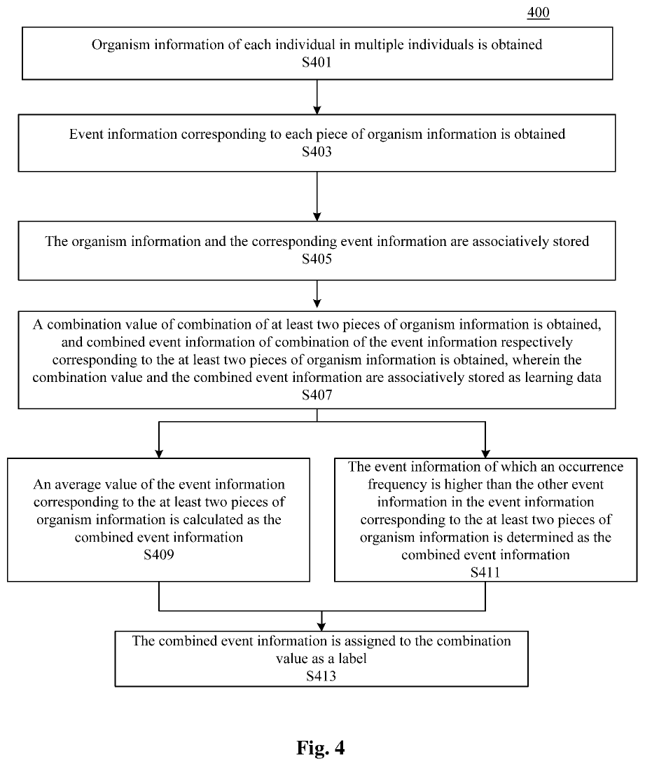

According to another embodiment of the present disclosure, a method for health management is provided. FIG. 4 is a flowchart of a method for health management according to an embodiment of the present disclosure. As shown in FIG. 4, the method for health management 400 includes the following steps. In S401, organism information of each person in multiple persons is obtained. In S403, event information corresponding to each piece of organism information is obtained. In S405, the organism information and the corresponding event information are associatively stored. In S407, a combination value of combination of at least two pieces of organism information is obtained, and combined event information of combination of the event information respectively corresponding to the at least two pieces of organism information is obtained, wherein the combination value and the combined event information are associatively stored as learning data. The method for health management according to the other embodiment of the present disclosure is the same as the method executed by the foregoing device for health management 200, and will not be elaborated herein.

In such a manner, the organism information and corresponding event information included in the learning data are combined and stored, so that it is impossible to directly obtain identification information, e.g. the original organism information, of objects or persons from the combined organism information.

As shown in FIG. 4, according to an exemplary embodiment of the present disclosure, the method for health management 400 further includes the following steps. In S409, an average value of the event information corresponding to the at least two pieces of organism information is calculated as the combined event information, wherein the event information is information in a numerical form. In S413, the combined event information is assigned to the combination value as a label. If the event information is, for example, information in the numerical form such as body weights, Step S409 is executed after Step S407 in the method for health management. Then, in Step S413, the combined event information is assigned to the combination value as the label.

Therefore, the event information in the numerical form is combined and processed, and it is impossible to directly obtain the identification information, used as the organism information, of the objects or the persons from the event information.

As shown in FIG. 4, according to an exemplary embodiment of the present disclosure, the method for health management 400 further includes the following step. In S411, the event information of which an occurrence frequency is higher than the other event information in the event information corresponding to the at least two pieces of organism information is determined as the combined event information, wherein the event information is information representing an action; and the combined event information is assigned to the combination value as a label. If the event information is information representing an action such as sitting, standing, walking and jumping, Step S411 is executed after Step S407 in the method for health management. Then, in Step S413, the combined event information is assigned to the combination value as the label.

Therefore, the event information representing the action is combined and processed, and it is impossible to directly obtain the identification information, used as the organism information, of the objects or the persons from the event information.

According to an exemplary embodiment of the present disclosure, the combination value is an average value of the at least two pieces of organism information. Therefore, the organism information is combined and processed, and it is impossible to directly obtain the identification information, used as the organism information, of the objects or the persons from the organism information.

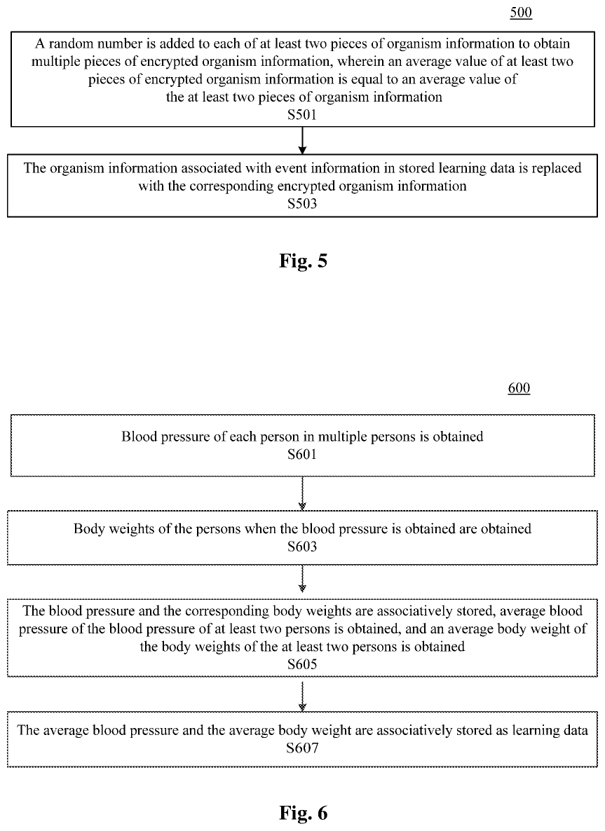

FIG. 5 is a flowchart of a method for health management according to an exemplary embodiment of the present disclosure. As shown in FIG. 5, the method for health management 500 includes the following steps. In S501, a random number is added to each of at least two pieces of organism information to obtain multiple pieces of encrypted organism information, wherein an average value of at least two pieces of encrypted organism information is equal to an average value of the at least two pieces of organism information. In S503, the organism information associated with event information in stored learning data is replaced with the corresponding encrypted organism information. In particular, the random numbers are generated when acquiring organism information from the persons and are used for encryption.

Therefore, the organism information is further encrypted, the organism information is changed at the same time of keeping the required learning data, and it is impossible to directly obtain identification information, such as organism information, of objects or persons from the organism information.

According to an exemplary embodiment of the present disclosure, at least two persons corresponding to the at least two pieces of organism information for combination are persons with the same event information. Therefore, the organism information suitable for the learning data is selected as learning data.

According to an exemplary embodiment of the present disclosure, for the at least two persons corresponding to the at least two pieces of organism information for combination, closeness between values of the at least two pieces of organism information is higher than a predetermined threshold value. Therefore, the organism information suitable for the learning data is selected as the learning data.

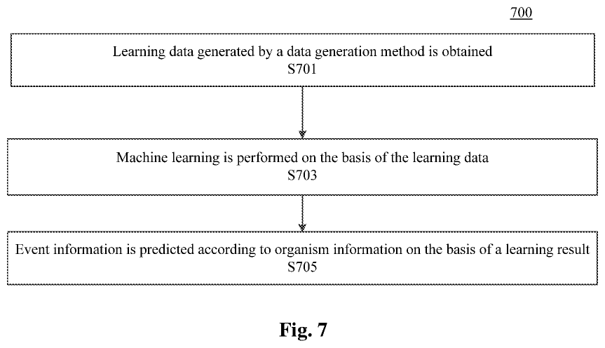

According to another aspect of the embodiments of the present disclosure, a method for health management is provided. FIG. 6 is a flowchart of a method for health management according to an exemplary embodiment of the present disclosure. As shown in FIG. 6, the method for health management 600 includes the following steps. In S601, blood pressure of each person in multiple persons is obtained. In S603, body weights of the persons when the blood pressure is obtained are obtained. In S605, the blood pressure and the corresponding body weights are associatively stored, average blood pressure of the blood pressure of at least two persons is obtained, and an average body weight of the body weights of the at least two persons is obtained. Wherein, in S607, the average blood pressure and the average body weight are associatively stored as learning data. In such a manner, the blood pressure and body weights of the persons included in the learning data are combined and stored, so that it is impossible to directly obtain identification information of the persons from the blood pressure and the body weights.

According to another aspect of the embodiments of the present disclosure, an event prediction method is provided. FIG. 7 is a flowchart of a method for health management according to an exemplary embodiment of the present disclosure. As shown in FIG. 7, the event prediction method 700 includes the following steps. In S701, learning data generated by the foregoing method for health management is obtained. In S703, machine learning is performed on the basis of the learning data. In S705, event information is predicted according to organism information on the basis of a learning result. Therefore, correct event prediction may be performed according to the processed learning data.

The method for health management according to the exemplary embodiment of the present disclosure is the same as the method executed by the device for health management 200 according to the embodiment of the present disclosure, and will not be elaborated herein.

According to another aspect of the embodiments of the present disclosure, a storage medium is provided, on which a program is stored, the program, when being executed, enabling equipment including the storage medium to execute the foregoing method.

According to another aspect of the embodiments of the present disclosure, a terminal is provided, which includes: one or more processors; a memory; a display device; and one or more programs, wherein the one or more programs are stored in the memory, and are configured to be executed by the one or more processors, and the one or more programs are configured to execute the foregoing method.

The program and storage medium according to the embodiments of the present disclosure refer to the contents mentioned above, and their specific implementation mode will not be elaborated herein. In the embodiments of the present disclosure, different emphases are laid to descriptions about each embodiment, and parts which are not elaborated in a certain embodiment may refer to the related descriptions in the other embodiments.

In the several embodiments provided in the present disclosure, it should be understood that the disclosed technical content may be implemented in other manners. The device embodiment described above is merely schematic. For example, the unit or module division may be a logic function division, and other division manners may be adopted during practical implementation. For example, a plurality of units or modules or components may be combined or may be integrated into another system, or some features may be ignored or not executed. In addition, the shown or discussed mutual coupling or direct coupling or communication connection may be indirect coupling or communication connection through some interfaces, units or modules, and may be in electrical or other forms.

The units or modules described as separate components may or may not be physically separated. The components displayed as units or modules may or may not be physical units or modules, that is, may be located in one place or may be distributed on multiple units or modules. Some or all of the units or modules may be selected according to actual needs to achieve the objectives of the solutions of the embodiments.

In addition, each of the functional units or modules in the embodiments of the present disclosure may be integrated in one processing unit or module, or each of the units or modules may exist physically and independently, or two or more units or modules may be integrated in one unit or module. The above-mentioned integrated unit or module may be implemented in form of hardware, and may also be implemented in form of software functional unit or module.

If being implemented in the form of a software functional unit and sold or used as an independent product, the integrated unit may be stored in a computer-readable storage medium. Based on this understanding, the technical solutions of the present disclosure essentially, or the part contributing to the prior art, or all or part of the technical solutions may be implemented in the form of a software product, and the computer software product is stored in a storage medium, including several instructions for causing a piece of computer equipment (such as a personal computer, a server or network equipment) to execute all or part of the steps of the method according to the embodiments of the present disclosure. The foregoing storage medium includes: various media capable of storing program codes such as a USB disk, a Read-Only Memory (ROM), a Random Access Memory (RAM), a removable hard disk, a magnetic disk, or an optical disk.

The foregoing is only the preferred embodiments of the present disclosure, and it should be noted that those of ordinary skilled in the art may make some improvements and modifications without departing from the principle of the disclosure. These improvements and modifications should be regarded to be within the scope of protection of the present disclosure.

### REFERENCE SIGNS IN THE ACCOMPANYING DRAWINGS

100: System for health management
110: Processor
112: Main memory
114: Memory
116: Input interface
118: Display interface
120: Communication interface
122: Bus
200: Device for health management
201: Acquisition unit
203: Input unit
205: Storage unit
207: Combination unit
209: Prediction unit
211: Label generation unit
213: Encrypted information generation unit

1. A device for predicting body weight of a person comprising:
an acquisition unit, configured to obtain blood pressure of each person in multiple persons;
an input unit, configured to input body weights of the persons when the blood pressure is obtained;
a storage unit, configured to associatively store the blood pressure and the corresponding body weights;
a combination unit, configured to obtain average blood pressure of the blood pressure of at least two persons and obtain an average body weight of the body weights of the at least two persons, wherein the average blood pressure and the average body weight are associatively stored in the storage unit as learning data, and
a prediction unit, configured to perform machine learning on the basis of the learning data and predict body weight according to blood pressure on the basis of a learning result.

2. A device for health management comprising:
an acquisition unit, configured to obtain organism information of each person in multiple persons;
an input unit, configured to obtain event information corresponding to each piece of organism information, the event information representing a physical entity parameter obtained by means of a sensing device;
a storage unit, configured to associatively store the organism information and the corresponding event information;
a combination unit, configured to obtain a combination value of combination of at least two pieces of organism information and obtain combined event information of combination of the event information respectively corresponding to the at least two pieces of organism information, wherein
the combination value and the combined event information are associatively stored in the storage unit as learning data, and
a prediction unit, configured to perform machine learning on the basis of the learning data and predict event information according to organism information on the basis of a learning result.

3. The device for health management of claim 2, wherein the event information is information in a numerical form, the device for health management further comprising:
a label generation unit, configured to calculate an average value of the event information corresponding to the at least two pieces of organism information as the combined event information; and
assign the combined event information to the combination value as a label.

4. The device for health management of claim 2, wherein the event information is information representing an action, the device for health management further comprising:
a label generation unit, configured to determine the event information of which an occurrence frequency is higher than the other event information in the event information corresponding to the at least two pieces of organism information as the combined event information; and
assign the combined event information to the combination value as a label.

5. The device for health management of claim 2, wherein
the combination value is an average value of the at least two pieces of organism information.

6. The device for health management of claim 5, further comprising:
an encrypted information generation unit, configured to add a random number to each of the at least two pieces of organism information to obtain multiple pieces of encrypted organism information, wherein an average value of at least two pieces of encrypted organism information is equal to the average value of the at least two pieces of organism information, and moreover,
the encrypted information generation unit sends the multiple pieces of encrypted organism information to the storage unit to replace the organism information associated with the event information in the stored learning data with the corresponding encrypted organism information.

7. The device for health management of claim 2, wherein
at least two persons corresponding to the at least two pieces of organism information for combination are persons with the same event information.

8. The device for health management of claim 2, wherein,
for the at least two persons corresponding to the at least two pieces of organism information for combination, closeness between values of the at least two pieces of organism information is higher than a predetermined threshold value.

9. A method for health management comprising:
obtaining organism information of each person in multiple persons;
obtaining event information corresponding to each piece of organism information, the event information representing a physical entity parameter obtained by means of a sensing device;
associatively storing the organism information and the corresponding event information; and
obtaining a combination value of combination of at least two pieces of organism information, and obtaining combined event information of combination of the event information respectively corresponding to the at least two pieces of organism information, wherein the combination value and the combined event information are associatively stored as learning data, and
performing machine learning on the basis of the learning data and predict event information according to organism information on the basis of a learning result.

10. The method for health management of claim 9, wherein the event information is information in a numerical form, the method for health management further comprising:
calculating an average value of the event information corresponding to the at least two pieces of organism information as the combined event information; and
assigning the combined event information to the combination value as a label.

11. The method for health management of claim 9, wherein
the event information is information representing an action, the method for health management further comprising:
determining the event information of which an occurrence frequency is higher than the other event information in the event information corresponding to the at least two pieces of organism information as the combined event information; and
assigning the combined event information to the combination value as a label.

12. The method for health management of claim 9, wherein
the combination value is an average value of the at least two pieces of organism information.

13. The method for health management of claim 12, further comprising:
adding a random number to each of the at least two pieces of organism information to obtain multiple pieces of encrypted organism information, wherein an average value of at least two pieces of encrypted organism information is equal to the average value of the at least two pieces of organism information, and moreover,
replacing the organism information associated with the event information in the stored learning data with the corresponding encrypted organism information.

14. The method for health management of claim 9, wherein
at least two persons corresponding to the at least two pieces of organism information for combination are persons with the same event information.

15. The method for health management of claim 9, wherein,
for the at least two persons corresponding to the at least two pieces of organism information for combination, closeness between values of the at least two pieces of organism information is higher than a predetermined threshold value.

16. The device for health management of claim 3, wherein
the combination value is an average value of the at least two pieces of organism information.

17. The device for health management of claim 4, wherein
the combination value is an average value of the at least two pieces of organism information.

18. The device for health management of claim 3, wherein
at least two persons corresponding to the at least two pieces of organism information for combination are persons with the same event information.

19. The device for health management of claim 4, wherein
at least two persons corresponding to the at least two pieces of organism information for combination are persons with the same event information.

20. The device for health management of claim 3, wherein,
for the at least two persons corresponding to the at least two pieces of organism information for combination, closeness between values of the at least two pieces of organism information is higher than a predetermined threshold value.
